(19) United States
(12) Patent Application Publication
(10) Pub. No.: US 2005/0112610 A1
(43) Pub. Date: May 26, 2005

(54) System and method for marking textiles with nucleic acids

(75) Inventors: Lawrence C. Lee, Los Angeles, CA (US); Donald Allen Alexander, Barnwell, SC (US); Jun Jei Shue, Chungho City (TW); Benjamin Liang, Chungho City (TW); Chung-Shung Chen, Chungho City (TW)

Correspondence Address: Michael A. Kerr;Virtual Legal, P.C., 777 E. William St., Suite 211, Carson City, NV, 89701, US

(73) Assignee: Applied DNA Sciences, Inc., Los Angeles, CA (US)

(21) Appl. No.: 10/825,968

(22) Filed: Apr. 15, 2004

Related U.S. Application Data
(60) Provisional application No. 60/463,215, filed on Apr. 16, 2003.

(51) Int. Cl. C12Q 1/68

(52) U.S. Cl. 435/6; 442/121

(57) ABSTRACT

A method for authenticating a textile material that is initiated by selecting a unique nucleic acid marker having a specific length and a specific sequence. A media that causes the unique nucleic acid marker to adhere to a fibrous material is then selected. The method then proceeds to generate a nucleic acid marker mixture by mixing the media with the nucleic acid marker. The nucleic acid marker mixture is then applied to the fibrous material. A marked fibrous material is produced by marking the fibrous material with the nucleic acid marker. The textile material is manufactured with the marked fibrous material. The textile material is then authenticated by detecting the unique nucleic acid marker with primers that are specific to the unique nucleic acid. In an alternative embodiment, the media is used as a topical treatment for the fibrous material. In another alternative embodiment, the media is a carrier media that can be added to one or more fiber manufacturing processes without affecting each of the manufacturing processes. In yet another alternative embodiment, a viscous solution for fiber spinning is selected and mixed with the nucleic acid marker to generate a viscous dope that is extruded through an opening in a spinneret to form a marked fiber that is used to generate the textile material.

### CROSS REFERENCE

This patent application is related to provisional patent application 60/463,215 which was filed on Apr. 16, 2003.

### BACKGROUND

1. Field of Invention

The invention is related to textiles. More particularly, the invention is related to marking textiles with nucleic acids.

2. Description of Related Art

With the dawn of the information age comes the ability to duplicate, change, alter and distribute just about anything. The FBI has called counterfeiting the crime of the 21^{st} century. Product counterfeiting is a serious and growing threat to brand names and labels within the textile industry. Measures to defend against counterfeiters and diverters are being taken by many corporations, but they have not developed comprehensive, systematic, and cost-effective solutions to preventing counterfeiting.

Thus there is a need within the textile industry to preserve and protect brand names. Brand names confer a substantial value on textile products. Consequently, brand names have become ripe targets for counterfeiters. For many companies, brand name equity represents its most important asset. These brand names have been built with enormous efforts and substantial investment. Nevertheless, these assets are vulnerable to the simplest forms of counterfeiting in today's international marketplace. The scale of product counterfeiting can only be estimated because of the difficulty in acquiring data. However, it is clear from both anecdotal evidence and available metrics that product counterfeiting is rapidly increasing.

Due to advancing counterfeiting techniques, traditional anti-counterfeit technologies are becoming obsolete. Additionally, governments and corporations that have invested a great deal of resources in fighting counterfeiting have experienced little or no success. Furthermore, law enforcement agencies that are burdened with efforts to combat violent crimes have insufficient resources to fight the “victimless” counterfeiting crime.

In addition to the counterfeiting concerns, foreign textile imports are threatening domestic textile companies. Recently enacted legislation attempts to restrict the flow of foreign textile imports. These safeguards would allow the national government to impose stiff tariffs or quotas to restrict the flow of certain foreign imports. As part of this legislation there is a need for marking domestic textile products so that domestic textile manufacturers can receive preferential tariff treatment.

### SUMMARY

A method for authenticating a textile material. The method is initiated by selecting a unique nucleic acid marker having a specific length and a specific sequence. A media that causes the unique nucleic acid marker to adhere to a fibrous material is then selected. The method then proceeds to generate a nucleic acid marker mixture by mixing the media with the nucleic acid marker. The nucleic acid marker mixture is then applied to the fibrous material. A marked fibrous material is produced by marking the fibrous material with the nucleic acid marker. The textile material is manufactured with the marked fibrous material. The textile material is then authenticated by detecting the unique nucleic acid marker with primers that are specific to the unique nucleic acid.

In an alternative embodiment, the media is used as a topical treatment for the fibrous material. In another alternative embodiment, the media is a carrier media that can be added to one or more fiber manufacturing processes without affecting each of the manufacturing processes. In yet another alternative embodiment, a viscous solution for fiber spinning is selected and mixed with the nucleic acid marker to generate a viscous dope that is extruded through an opening in a spinneret to form a marked fiber that is used to generate the textile material.

### BRIEF DESCRIPTION OF THE DRAWINGS

Embodiments for the following description are shown in the following drawings:

FIG. 1 is a flowchart of a method for authenticating textiles.

FIG. 2 is a flowchart of an illustrative textile manufacturing process having a variety of insertion points for the nucleic acid marker.

FIG. 3 is a flowchart of an illustrative method for embedding the nucleic acid marker into a fibrous material.

FIG. 4 is a flowchart of an illustrative method for applying a nucleic acid marker to identify the origin of a yarn and/or thread.

FIG. 5 is a flowchart of an illustrative method for applying the nucleic acid marker during an ink mixing process.

### SPECIFICATION

In the following detailed description, reference is made to the accompanying drawings, which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural and logical changes may be made without departing from the spirit and scope of the claims. The following detailed description is, therefore, not to be taken in a limited sense.

Note, the leading digit(s) of the reference numbers in the Figures corresponds to the figure number, with the exception that the same reference numbers identifies identical components, which appear in multiple figures.

A textile authentication nucleic acid marker method described herein may be applied to fibers, yarns, sewing thread, fabrics, non-woven materials, and all products made from fibrous materials. The products made from fibrous materials include apparel, home technical automotive, medical, aerospace, consumer products and other such products.

Fibers are any substance, natural or manufactured, with a high length-to-width ratio and with suitable characteristics for being processed into fabric in which the smallest component is hairlike in nature and can be separated from a fabric. Natural fibers are those that are in a fiber form as they grow or develop and come from animal, plant, or mineral sources. Manufactured fibers are made from chemical compounds produced in manufacturing facilities. The first manufactured fiber was Rayon.

Yarns are an assemblage of fibers that are twisted or laid together so as to form a continuous strand that can be made into textile fabric. A yarn is a continuous strand of textile fibers, filaments, or materials in a form suitable for knitting, weaving, or otherwise intertwining to form a textile fabric. Filament yarns are made from manufactured fibers, except for a tiny percentage that is filament silk. Manufactured filament yarns are made by extruding a polymer solution through a spinneret, solidifying it in fiber form, and then bringing the individual filaments together with or without a twist. Spun yarns are continuous strands of staple fibers held together by some mechanism such as a mechanical twist that uses fiber irregularities and natural cohesiveness to bind the fibers together into one yarn.

Sewing thread is a yarn intended for stitching materials together using machine or hand processes.

Fabric is flexible planar substance constructed from solutions, fibers, yarns, or fabrics, in any combination. A fabric is a pliable, planelike structure that can be made into two- or three-dimensional products that require some shaping and flexibility. Fabrics can be made from a wide variety of starting materials: solutions, fibers, yarns and “composite” fabrics. For fabrics made from yarns, the fabric is either woven or knitted fabrics. With the exception of triaxial fabrics, all woven fabrics are made with two or more sets of yarns interlaced at right angles. Knitting is the formation of a fabric by the interlooping of one or more sets of yarns. Fabrics from solutions include films in which the films are made directly from a polymer solution by melt extrusion or by casting the solution onto a hot drum. Composite fabrics are fabrics that combine several primary and/or secondary structures, at least one of which is a recognized textile structure, into a single structure.

Some fabrics are made directly from fibers or fiber forming solutions and there is no processing of fibers into a yarn. These nonwoven structures include all textile-sheet structures made from fibrous webs, bonded by mechanical entanglement of the fibers or by the use of added resins, thermal fusion, or formation of chemical complexes.

Selection and Detection of Nucleic Acid Marker

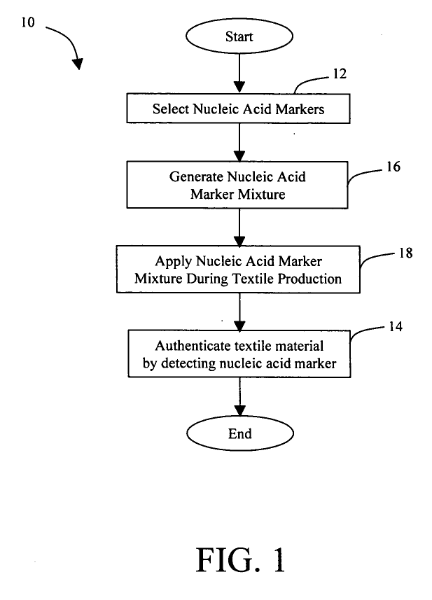

Referring to FIG. 1, there is shown a method 10 for authenticating a textile material. The method is initiated at block 12 by selecting a unique nucleic acid (NA) marker. The term nucleic acid, which is also abbreviated as “NA” in the Figures, is a general term for deoxyribonucleic acid (DNA) or ribonucleic acid (RNA). The nucleic acid can be chosen from animals, plants, bacteria, viruses, fungi, or synthetic vectors or fragments or any combination thereof. By way of example and not of limitation, the unique nucleic acid marker may be obtained from Biowell Technologies, Inc. of Taiwan.

For the illustrative embodiment, the unique nucleic acids have a specific length and a specific length, so that when polymerase chain reaction (PCR) procedures are performed, only PCR primers with correct sequences can produce the original nucleic acid. Additionally, there is a low concentration of unique nucleic acids within the collection sample that makes it difficult to decode the unique nucleic acids through cloning and transgenic methods. Thus, during the authentication of the textile materials in block 14, the use of low concentrations of unique nucleic acids in combination with the specificity associated with the use of specific PCR primers results in a unique nucleic acid sequence that is extremely difficult to copy.

The authenticated unique nucleic acid marker also has the added benefit of being used to identify specific characteristics of the textile material. The specific characteristics of the textile material includes a variety of product information. By way of example and not of limitation, the product information may comprise country of origin for the textile material, origin of the final product, information about the manufacturer, plant identification, product identification and other related data.

The illustrative unique nucleic acid marker in block 14 is authenticated using test kits, portable scanners and lab verification. By way of example and not of limitation, the test kits, portable scanners and lab verification may be purchased and/or performed by Biowell Technology Inc. For illustrative purposes only, the identification data for each nucleic acid marker is stored in a database. This database comprises a plurality of product information as described above.

For the illustrative authentication in block 14 there are two authentication levels. The first authentication level is performed with an infrared scanner and the results are immediate. The infrared scanner comes pre-loaded and is not tethered to a database. Any needed updates can be made during regular maintenance. The second authentication level is the in-depth authentication testing in which the nucleic acid marker is detected with a suitcase sized test kit using the PCR primers described above. In one illustrative embodiment, the in-depth authentication testing takes approximately 20 to 30 minutes with the suitcase size test kit.

The recovery solvent used during the in-depth authentication process has a high nucleic acid solubility and extracts the unique nucleic acid. The recovery solvent may utilize organic or inorganic solvents for extraction. By way of example and not of limitation, the organic solvent may be a buffer, benzene, characin, alcohol, acetone, or chloroform. The buffer may be a phosphate based buffer. By way of example and not of limitation, the inorganic solver is water.

Well known PCR amplification procedures are used to examine the authenticity of the nucleic acid. The PCR methods may be single or multiple nested PCR. If the examined object carries the original nucleic acid, the PCR procedure will amplify the extracted nucleic acid several million times with the same size and sequence of the original nucleic acid. If the examined object does not have the original nucleic acid, there will be no amplified nucleic acid product. Therefore, by comparing the size and amount of PCR products, the authenticity of labeled objects can be verified.

By way of example and not of limitation, the authentication may be performed at the borders by an authority such as the United States Customs and Border Protection. The authentication process may also be performed by a qualified laboratory such as Biowell Technologies, Inc.

Generating a Nucleic Acid Marker Mixture for Textile Applications

After selecting the unique nucleic acid marker, the method proceeds to block 16. At block 16 a nucleic acid marker mixture is generated by mixing the nucleic acid marker with a media that is selected for its particular properties. In a first illustrative embodiment, a media is selected that causes the nucleic acid marker to adhere to a fibrous material. The media is then mixed with the nucleic acid marker to generate a nucleic acid marker mixture. The nucleic acid marker mixture is then applied to a fiber or a fibrous material. As a result of this application, a marked fibrous material is generated by causing the nucleic acid marker to adhere to the fibrous material. By way of example and not of limitation, the media for the first illustrative embodiment is selected from a group consisting of aqueous solvents, adhesives, polymers, binders, or cross-linking agents. Another illustrative example of the media for the first illustrative embodiment is selected from the group consisting of acrylic, polyurethane, dimethlyoldihydroxyethyleneurea, polyvinyl alcohol, starch, epoxy, or polyvinyl chloride.

In a second illustrative embodiment, a media is selected that is used as a topical treatment for a fibrous material. The media is then mixed with the nucleic acid marker to generate a nucleic acid marker mixture. The nucleic acid marker mixture is then applied to the fibrous material. A marked fibrous material is then generated by causing the nucleic acid marker to adhere to the fibrous material. The media for the for the second illustrative embodiment is selected from a group consisting of colorants, dyes, dyeing auxiliaries, print pastes, softeners, lubricants, antistatic agents, water repellants, moisture transport, soil resistance, antimicrobial agents, wetting agents, leveling agents, or water.

In a third illustrative embodiment, a carrier media is selected that can be added to one or more of a plurality of fiber manufacturing processes without affecting each of the fiber manufacturing processes. The method then proceeds to mix the carrier media with the unique nucleic acid to generate a nucleic acid mixture. The nucleic acid marker mixture is applied to the fibrous material to generate a marked fibrous material in which the nucleic acid marker adheres to the fibrous material.

In a fourth illustrative embodiment, the media is a viscous spinning solution for fiber spinning. The viscous spinning solution is mixed with the nucleic acid marker to generate a viscous dope having the unique nucleic acid marker. The viscous dope is then extruded through an opening in a spinneret to form a marked fiber. The marked fiber is then solidified and can then be used in the textile manufacturing process. With this method the nucleic acid marker mixture is embedded in the fiber.

In a fifth embodiment, the unique nucleic acid is mixed with a water insoluble media to generate the nucleic acid marker mixture. Firstly, the unique nucleic acid is dissolved in a water soluble solution. The method then proceeds to dissolve the water insoluble media in a solvent. An intermediate solution is then used to mix the water soluble solution having the nucleic acid marker with the water insoluble media. The resulting nucleic acid marker mixture is then applied to the desired object. By way of example and not of limitation, the water insoluble medium is selected from a group consisting of polymer materials such as polypropylene, polycarbonate, or polystyrene. By way of example and not of limitation, the intermediate solution used to generate the nucleic acid marker mixture is an organic solvent such as ethanol, acetone, chloroform or other such organic mixtures.

After block 16, the method proceeds to block 18 in which the nucleic acid marker mixture is inserted into a textile manufacturing process. There are a number of insertion points that can be used for inserting the nucleic acid marker mixture. A plurality of different insertion points are described in further detail below. The insertion of the nucleic marker mixture also includes “embedding” the nucleic acid marker into a fiber or fibrous material to produce a marked fiber.

Illustrative Applications of Nucleic Acid Marker to Textiles

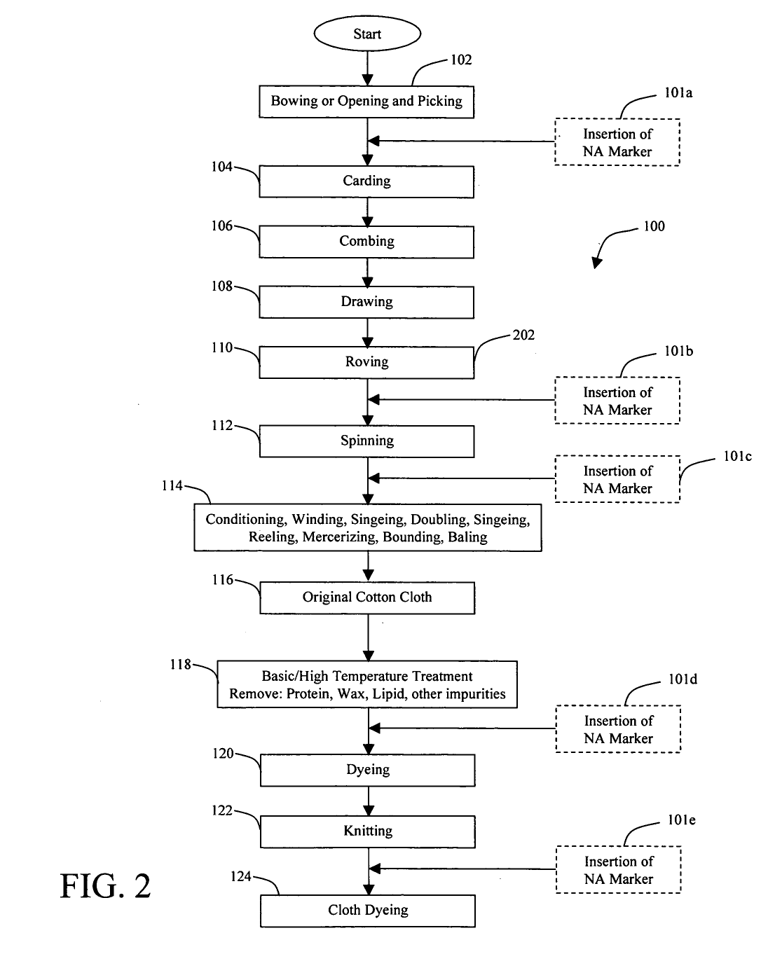

Referring to FIG. 2 there is shown an illustrative textile manufacturing process 100 having a variety of insertion points for the nucleic acid marker. The nucleic acid marker is applied as a nucleic acid marker mixture as described above. The illustrative insertion points 101a, 101b, 101c, 101d, and 101e for the nucleic acid marker mixture provide for the application of the nucleic acid marker mixture during the illustrative textile manufacturing process. During the textile manufacturing process, one or more nucleic acid marker mixtures may be inserted at one or more insertion points. An operational database is maintained to register each of the nucleic acid sequences for each manufacturer or process using the textile manufacturing process.

The first insertion point 101a occurs after the bowling or opening and picking process 102. The illustrative method then proceeds to the process steps of carding 104 during which staple fibers are drawn together in a somewhat parallel arrangement to form a very weak rope of fibers. The method continues to combing 106 which is an additional step in the production of smooth, fine, uniform spun yarns made of long-staple fibers. The next step is drawing 108 in which a manufactured fiber is elongated after spinning to alter the molecular arrangement within the fiber. During roving 110, the drawn sliver is reduced in size, fiber are made more parallel, and a small amount of twist is inserted.

The second illustrative insertion point 101b for the nucleic acid marker mixture takes place after the roving 110 process and before spinning 112. Spinning refers to the process of producing yarn from staple fibers, it also refers to the production of a fiber by extruding a solution through tiny holes in a spinneret.

The third illustrative insertion point 101c occurs after spinning 112 and before block 116. In block 116, the illustrative following steps occur, namely, conditioning, winding, singeing, doubling, singeing, reeling, mercerizing, bounding and baling. Winding refers to the process of transferring yarn of transferring from one package to another. Singeing burns the fiber ends from the fabric to produce a smooth surface. Reeling refers to the process of removing fibers and winding them into a reel. Mercerization is a finish in which sodium hydroxide is used to increase cotton's absorbency, luster and strength.

The method then proceeds to block 116 in which the original cotton cloth is generated. After the original cotton cloth is generated, the method proceeds to block 118 in which a basic high temperature treatment takes place that removes, proteins, wax, lipids and other impurities.

The illustrative fourth insertion point 101d occurs after the basic high temperature treatment and before the dyeing block 120. The dyeing process block 120 refers, in general, to the addition of color to the illustrative textile manufacturing process. Most colored textiles are produced by the use of dye or pigment mixtures. Pigments are insoluble color particles that are held on the surface of a fabric by a binding agent. Dye is an organic compound composed of a colored portion and includes a site that permits bonding to the fiber. Thus, for the illustrative fourth insertion point the nucleic acid marker may be combined with a dye mixture or pigment mixture prior to attachment of the nucleic acid market to the textile.

After dyeing block 120, the method proceeds to knitting block 122. Knitting refers to the process of fabric production by interlooping yarns. The illustrative fifth insertion point 101e occurs after knitting block 122 and before cloth dyeing block 124. In the illustrative textile manufacturing process 100, the cloth dyeing process is performed after knitting so that the knitted textile may be colored again. As previously described, the nucleic acid marker may be combined with a dye mixture or pigment mixture prior to attachment to the textile.

During the first three insertion points, namely 101a, 101b, 101c, the nucleic acid marker mixture is applied directly to a fiber or a fibrous material. As described above, the nucleic acid marker is blended with a media that generates a nucleic acid marker mixture that will cause the nucleic marker to adhere to a fibrous material or to products made from fibrous materials. The media causes the nucleic acid marker to adhere to the fibrous material or to products made from fibrous materials.

An illustrative example of media which causes the nucleic acid marker to adhere to the fibrous material or to products made from fibrous materials includes but is not limited to adhesives, polymers, binders, cross-linking agents. For example, the media may be an aqueous solvent such as acrylic, polyurethane, dimethyloldihydroxyethyleneurea (DMDHEU), polyvinyl alcohol, starch, epoxy, or polyvinyl chloride (PVC). Additionally, the media may be a dry adhesive or polymer. Furthermore, as previously described the media may have a variety of characteristics such as being a water insoluble media or a water soluble media.

With respect to the fourth and fifth insertion points, namely, insertion points 101d and 101e, the insertion points are performed in what is generally referred to as the “finishing” processes. A finishing process is any process used to add color and augment performance of unfinished fabric. A finish is any process that is performed to fiber, yarn, or fabric either before or after fabrication to change the appearance (what is seen), the hand (what is felt), or the performance (what the fabric does).

Thus, the nucleic acid marker is mixed with a media that is used as a topical treatment and/or finishing treatment for fibers, fibrous materials, and products made from fibrous materials. Such media are commonly used as colorants or various finishes including dyeing auxiliaries, print pastes, softeners, lubricants, antistatic agents, water repellants, moisture transport, soil resistance, antimicrobial, wetting agents, leveling agents, water, etc.

The method for generating a nucleic acid marker mixture for insertion in the textile manufacturing process may be performed in a variety of different ways. In one illustrative embodiment, the nucleic acid marker mixture comprises the step of mixing the unique nucleic acid sequence with a first media that is liquefied in a solvent. The nucleic acid marker mixture is then applied to the textile. The first media solidifies after the evaporation of the solvent.

Another illustrative technique is to mix the unique nucleic acid with a water insoluble media to generate the nucleic acid marker mixture. In this second technique, the unique nucleic acid is first dissolved in a water soluble solution. Then the water insoluble media is dissolved in a solvent. An intermediate solution is then used to mix the water soluble solution having the nucleic acid marker with the water insoluble media. The resulting nucleic acid marker mixture is then applied to the textile. The water insoluble media is used to introduce the nucleic acid marker to various “host chemical” systems or water baths without interfering with the properties of the “host chemical” system. Those skilled in the art shall appreciate that the “host chemical” system may be used in the process or treatment of fibrous materials or products made from fibrous materials.

Yet another technique to generate the nucleic acid marker mixture is to provide a chemically active surface on the nucleic acid marker. The chemically active surface is then directly reacted with a fibrous material or a treatment applied to the fibrous material. By way of example and not of limitation, a reaction site on the nucleic acid marker is generated and then reacts with cellulose (cotton fiber, etc.). Additionally, the reaction site on the nucleic acid marker may also react with nylon, certain polyesters, wool, or other fiber types.

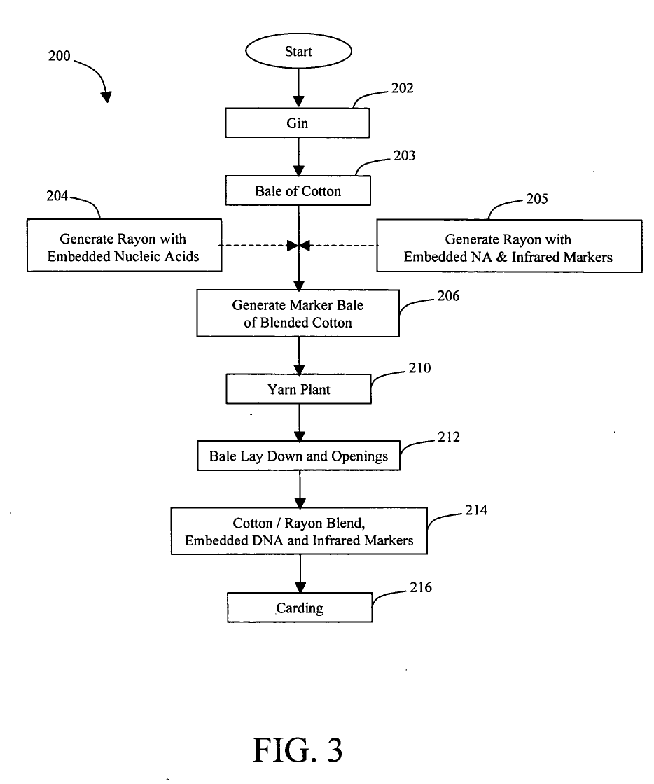

Referring to FIG. 3 there is shown another illustrative method 200 for embedding the nucleic acid marker into a fibrous material. The nucleic acid marker mixture is embedded into fibrous materials during the manufacturing of the fibers or fibrous materials. The illustrative method 200 is initiated a block 202 in which a gin is used to separate the cotton fibers from the seed. The method then proceeds to block 203 in which a bale of cotton is produced.

At block 204, the nucleic acid marker is embedded into a fiber such as rayon. Alternatively, an infrared marker and nucleic acid marker may be embedded into the illustrative rayon fiber as described in block 205. The nucleic acid marker is embedded into the fibers or fibrous materials using additional processing equipment, chemistry, and conditions as necessary to embed the nucleic acid marker into the fibrous materials or products made from fibrous materials.

The rayon is then blended with the cotton from the bale of cotton to generate a marker bale of blended cotton as described in block 206. The resulting “blend” is an intimate mixture of fibers of different generic type, composition, length, diameter, or color spun together in one yarn. In intimate blends, both fibers are present in the same yarn in planned proportions. Fiber types cannot be separated; they are next to each other throughout the yarn.

The method then proceeds to block 210 in which the marker bale is then received by a yarn plant. The bale proceeds to the lay down and opening process in block 212. Opening is an initial step in the production of spun yarns which loosens fibers from the bale form and cleans and blends the fibers. Thus the treated cotton fibers referred to as “marked fibers” are combined with other cotton fibers to generate a blend of combined cotton that can be identified using the nucleic acid markers.

The illustrative method then proceeds to the carding process in block 216. During carding stable fibers are drawn together in a somewhat parallel arrangement to form a weak rope of fibers referred to as a “carded sliver.”

0After carding, the fibers or fibrous materials that have been treated with the nucleic acid markers may be further combined to produce a yarn, thread, fabric, nonwoven fabric, or any product made using fibrous materials. By way of example and not of limitation, the illustrative yarn containing the nucleic markers may be combined with one or more yarns that do not contain nucleic acid markers. The resulting product would have the capability of being identified by the nucleic markers in the embedded rayon from block 204 and block 205.

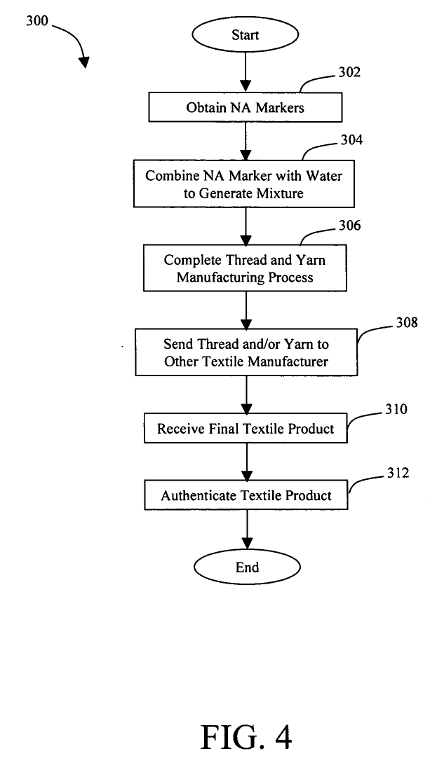

Referring to FIG. 4 there is shown yet another illustrative method 300 for applying a nucleic acid marker to identify the origin of a yarn and/or thread. The method is initiated at block 302 in which the nucleic acid markers are obtained. The nucleic acid markers are associated with a manufacturer. Using one of the methods described above, the nucleic acid marker is combined with an illustrative media such as water to generate a nucleic acid marker mixture.

During the thread and yarn manufacturing process of block 306, the nucleic acid marker mixture is sprayed on an illustrative cotton fiber during the illustrative bale opening process. For the illustrative method, the nucleic acid marker is not affected by the downstream textile manufacturing process.

At block 308, the thread and/or yarn is sent to an overseas textile manufacturer for further processing. At block 310, the finished textile is received in the illustrative country of origin. At block 312, the authentication methods described above are used to confirm that the illustrative cotton thread and/or yarn was manufactured in the country of origin.

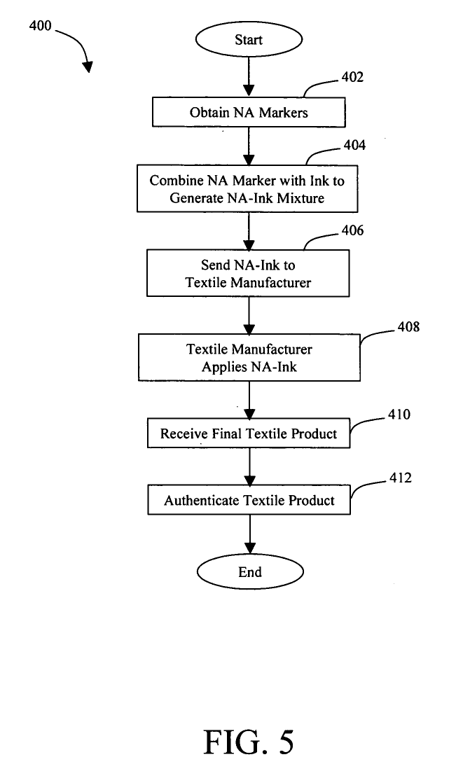

Referring to FIG. 5 there is shown yet another illustrative method for applying the nucleic acid marker during an ink mixing process. The method is initiated at block 402 with the obtaining of nucleic acid markers that are related to the textile manufacturer. At block 404, the nucleic acid marker is combined with ink to generate a nucleic acid ink mixture. As described in block 406, the nucleic acid ink mixture is then sent to the textile manufacturer. The textile manufacturer then proceeds to apply the nucleic acid ink mixture as shown in block 408. At block 410, the finished textile is received. At block 312, the authentication methods described above is use to verify that the textile has the appropriate nucleic acid marker.

It shall be appreciated by those skilled in the art having the benefit of this disclosure that the process of marking fibrous materials or products made from fibrous materials using the nucleic acid markers may be used to identify specific characteristic of the marked materials or products. By way of example and not of limitation, the nucleic acid marker may be embedded in a sewing thread that is associated with a particular manufacturer that only uses the marked sewing thread. This type of application could be used to determine the origin and other supply information of the textile.

It shall also be appreciated by those of ordinary skill in the art that the nucleic acid marker may be combined with an one or more infrared markers. The mixture of the nucleic acid marker, infrared marker, and the media may be combined to generate a marker mixture that is applied to one or more fibers or fibrous materials. The marked fibers may then be blended with one or more unmarked fibers to generate the marked textile. The blending of the marked first fiber with the unmarked fiber can be performed during ginning, before opening, during opening, before blending, during blending. Additionally the nucleic acid markers and infrared markers can be mixed in a dyeing process. By way of example and not of limitation, the marked fiber may comprise rayon.

Additionally, those skilled in the art shall appreciate that the systems and methods described above may be used to mark materials, packaging, labeling, documents, and shipping containers for determining the origin, authenticity, or other supply chain or product information.

It shall be appreciated by those of ordinary skill in the art that the functions described above may be customized depending on particular requirements and the level of security and authentication required. Additionally, alternate embodiments of the invention will be apparent to those skilled in the art. Although the description above contain many limitations, these should not be construed as limiting the scope of the claims but as merely providing illustrations of some of the presently preferred embodiments of this invention. Many other embodiments will be apparent to those of skill in the art upon reviewing the description. Thus, the scope of the invention should be determined by the appended claims, along with the full scope of equivalents to which such claims are entitled.

1. A method for authenticating a textile material, comprising:
selecting a unique nucleic acid marker having a specific length and a specific sequence;
selecting a media that causes said unique nucleic acid marker to adhere to a fibrous material;
mixing said media with said nucleic acid marker to generate a nucleic acid marker mixture;
applying said nucleic acid marker mixture to said fibrous material;
generating a marked fibrous material by causing said nucleic acid marker to adhere to said fibrous material;
producing said textile material by using one or more fibrous materials wherein one of said plurality of fibrous materials is said marked fibrous material; and
authenticating said textile material by detecting said unique nucleic acid marker in said marked fibrous material, said nucleic acid detected with primers particular to said unique nucleic acid having said specific length and said specific sequence.

2. The method of claim 1 wherein said media is selected from a group consisting of aqueous solvents, adhesives, polymers, binders, or cross-linking agents.

3. The method of claim 1 wherein said media is selected from a group consisting of acrylic, polyurethane, dimethyloldihydroxyethyleneurea, polyvinyl alcohol, starch, epoxy, or polyvinyl chloride.

4. The method of claim 1 wherein said textile material is selected from a textile group consisting of yarns, sewing threads, fabrics, nonwoven materials, or products manufactured from fibrous materials.

5. The method of claim 4 wherein said plurality of products manufactured from fibrous materials is selected from a group consisting of apparel, home, technical, automotive, medical, aerospace, or consumer products.

6. The method of claim 1 wherein said nucleic acid is deoxyribonucleic acid.

7. The method of claim 1 wherein said nucleic acid is ribonucleic acid.

8. The method of claim 1 wherein said authenticating of said textile material further comprises identifying specific characteristics of said textile material.

9. The method of claim 8 wherein said identifying specific characteristics of said textile material further comprises determining a plurality of product information about said textile material.

10. The method of claim 9 wherein said product information is selected from a group consisting of product origin, supply chain information, or manufacturing information.

11. A method for authenticating a textile material, comprising:
selecting a unique nucleic acid marker having a specific length and a specific sequence;
selecting a media that is used as a topical treatment for a fibrous material;
mixing said media with said nucleic acid marker to generate a nucleic acid marker mixture;
applying said nucleic acid marker mixture to said fibrous material;
generating a marked fibrous material by causing said nucleic acid marker to adhere to said fibrous material;
producing said textile material by using one or more fibrous materials wherein one of said plurality of fibrous materials is said marked fibrous material; and
authenticating said textile material by detecting said unique nucleic acid marker in said marked fibrous material, said nucleic acid detected with primers particular to said unique nucleic acid having said specific length and said specific sequence.

12. The method of claim 11 wherein said media is selected from a group consisting of colorants, dyes, dyeing auxiliaries, print pastes, softeners, lubricants, antistatic agents, water repellants, moisture transport, soil resistance, antimicrobial, wetting agents, leveling agents, or water.

13. The method of claim 11 wherein said textile material is selected from a textile group consisting of yarns, sewing threads, fabrics, nonwoven materials, or products manufactured from fibrous materials.

14. The method of claim 13 wherein said plurality of products manufactured from fibrous materials is selected from a group consisting of apparel, home, technical, automotive, medical, aerospace, or consumer products.

15. The method of claim 11 wherein said nucleic acid is deoxyribonucleic acid.

16. The method of claim 11 wherein said nucleic acid is ribonucleic acid.

17. The method of claim 11 wherein said authenticating of said textile material further comprises identifying specific characteristics of said textile material.

18. The method of claim 17 wherein said identifying specific characteristics of said textile material further comprises determining a plurality of product information about said textile material.

19. The method of claim 18 wherein said product information is selected from a group consisting of product origin, supply chain information, or manufacturing information.

20. A method for authenticating a textile material, comprising:
selecting a unique nucleic acid marker having a specific length and a specific sequence;
selecting a carrier media that can be added to one or more of a plurality of fiber manufacturing processes without affecting each of said fiber manufacturing processes;
mixing said carrier media with said nucleic acid marker to generate a nucleic acid marker mixture;
applying said nucleic acid marker mixture to said fibrous material;
generating a marked fibrous material by causing said nucleic acid marker to adhere to said fibrous material;
producing said textile material by using one or more fibrous materials wherein one of said plurality of fibrous materials is said marked fibrous material; and
authenticating said textile material by detecting said unique nucleic acid marker in said marked fibrous material, said nucleic acid detected with primers particular to said unique nucleic acid having said specific length and said specific sequence.

21. The method of claim 20 wherein said textile material is selected from a textile group consisting of yarns, sewing threads, fabrics, nonwoven materials, or products manufactured from fibrous materials.

22. The method of claim 21 wherein said plurality of products manufactured from fibrous materials is selected from a group of products manufactured from fibrous materials consisting of apparel, home, technical, automotive, medical, aerospace, or consumer products.

23. The method of claim 20 wherein said nucleic acid is deoxyribonucleic acid.

24. The method of claim 20 wherein said nucleic acid is ribonucleic acid.

25. The method of claim 20 wherein said authenticating of said textile material further comprises identifying specific characteristics of said textile material.

26. The method of claim 25 wherein said identifying specific characteristics of said textile material further comprises determining a plurality of product information about said textile material.

27. The method of claim 26 wherein said product information is selected from a group consisting of product origin, supply chain information, or manufacturing information.

28. A method for authenticating a textile material, comprising:
selecting a unique nucleic acid marker having a specific length and a specific sequence;
selecting a viscous spinning solution for fiber-spinning;
mixing said viscous spinning solution with said nucleic acid marker to generate a viscous dope having said unique nucleic acid marker;
extruding said viscous dope through an opening in a spinneret to form a marked fiber;
solidifying said marked fiber;
producing said textile material by using one or more fibrous materials wherein one of said plurality of fibrous materials is said marked fiber; and
authenticating said textile material by detecting said unique nucleic acid marker in said marked fiber, said nucleic acid detected with primers particular to said unique nucleic acid having said specific length and said specific sequence.

29. The method of claim 28 wherein said viscous spinning solution is selected from a group consisting of acetate, rayon, acrylic, nylon, polyester, or glass.

30. The method of claim 28 wherein said textile material is selected from a textile group consisting of yarns, sewing threads, fabrics, nonwoven materials, or products manufactured from fibrous materials.

31. The method of claim 30 wherein said plurality of products manufactured from fibrous materials is selected from a group consisting of apparel, home, technical, automotive, medical, aerospace, or consumer products.

32. The method of claim 30 wherein said nucleic acid is deoxyribonucleic acid.

33. The method of claim 30 wherein said nucleic acid is ribonucleic acid.

34. The method of claim 30 wherein said authenticating of said textile material further comprises identifying specific characteristics of said textile material.

35. The method of claim 34 wherein said identifying specific characteristics of said textile material further comprises determining a plurality of product information about said textile material.

36. The method of claim 35 wherein said product information is selected from a group consisting of product origin, supply chain information, or manufacturing information.

37. A method for manufacturing a marked textile to authenticate said marked textile's origin, comprising:
providing at least one nucleic acid marker;
mixing said at least one nucleic acid marker with a liquid;
spraying said liquid on a first fiber so as to mark said first fiber with nucleic acid; and
combining said marked first fiber with one or more unmarked fibers to generate said marked textile.

38. The method of claim 37 wherein said spraying of said liquid is performed during a bale opening process.

39. The method of claim 37 wherein said spraying of said liquid is performed during a knitting/weaving process.

40. The method of claim 37 wherein said liquid includes an ink that is used during a dyeing process.

41. The method of claim 37 wherein after combining said marked first fiber with one or more unmarked fibers, the method further comprises processing said marked textile using typical textile processes.

42. The method of claim 37 wherein said first fiber comprises rayon.

43. The method of claim 37 wherein said first fiber is configured to adhere to said at least one nucleic acid marker.

44. The method of claim 37 further comprising mixing said liquid in a dyeing process.

45. A method for manufacturing a marked textile to authenticate said marked textile's origin, comprising:
providing at least one nucleic acid marker;
providing an infrared marker;
embedding said at least one nucleic acid marker and said infrared marker into a first fiber so as to mark said first fiber;
blending said marked first fiber with one or more unmarked fibers to generate said marked textile.

46. The method of claim 45 wherein said blending of said marked first fiber with one or more unmarked fibers is performed during ginning.

47. The method of claim 45 wherein said blending of said marked first fiber with one or more unmarked fibers is performed before opening of a yarn manufacturing process.

48. The method of claim 45 wherein said blending of said marked first fiber with one or more unmarked fibers is performed during opening of a yarn manufacturing process.

49. The method of claim 45 wherein said blending of said marked first fiber with one or more unmarked fibers is performed before blending of a yarn manufacturing process.

50. The method of claim 45 wherein said blending of said marked first fiber with one or more unmarked fibers is performed during blending of a yarn manufacturing process.

51. The method of claim 45 wherein said first fiber comprises rayon.

52. The method of claim 45 wherein said first fiber is configured to adhere to said at least one nucleic marker.

53. The method of claim 45 further comprising mixing said at least one nucleic markers in a dyeing process for yarn manufacturing.
